(19) United States
(12) Patent Application Publication
(10) Pub. No.: US 2026/0187115 A1
(43) Pub. Date: Jul. 2, 2026

(54) METHOD FOR OPTIMIZING LARGE LANGUAGE MODEL CONVERSATION, APPARATUS, DEVICE, AND COMPUTER STORAGE MEDIUM

(71) Applicant: Beijing Yilian Zhishu Technology Co., Ltd., Beijing (CN)

(72) Inventor: Shirui WANG, Beijing (CN)

(73) Assignee: Beijing Yilian Zhishu Technology Co., Ltd., Beijing (CN)

(21) Appl. No.: 19/411,589

(22) Filed: Dec. 8, 2025

(30) Foreign Application Priority Data
Dec. 30, 2024 (CN) 202411978725.8

(51) Int. Cl. G06F 16/3329

(52) U.S. Cl. CPC G06F 16/33295

(57) ABSTRACT

The present disclosure provides a method for optimizing large language model conversation, including: in response to entering a current conversation turn, acquiring configuration information corresponding to the current conversation turn based on a type of the current conversation turn; acquiring user input information corresponding to the current conversation turn; determining whether the current conversation turn needs to be switched based on a conversation turn switching determination strategy corresponding to the conversation turn switching strategy configuration information; when the current conversation turn needs to be switched, determining a next conversation turn corresponding to the current conversation turn, and entering the next conversation turn; when the current conversation turn does not need to be switched, generating a response task, and performing the response task calling a large language model to generate a response message; and outputting the response message in the current conversation turn, to meet user expectations.

### CROSS-REFERENCE TO RELATED APPLICATIONS

This application claims priority of Chinese Patent Applicant No. 202411978725.8, filed on Dec. 30, 2024, entitled as “Method for Optimizing Large Language Model Conversation, Apparatus, Device, and Computer Storage Medium,” the entire disclosure of which is incorporated herein by reference for all purposes.

### TECHNICAL FIELD

The present disclosure relates to the field of language models, and particularly to a method for optimizing large language model conversation, an apparatus, a device, and a computer storage medium.

### BACKGROUND

In the field of natural language processing, a Large Language Model (LLM) is an artificial intelligence model that, through training on massive amounts of data, can understand and generate natural language. It has been widely applied in various scenarios, such as intelligent customer service and online consultation.

LLMs in the related art typically use preset prompts to guide conversations. These prompts are fixed during training and lack the ability to be dynamically generated or adjusted based on user input. Therefore, they are not flexible enough when handling diverse user needs.

### SUMMARY

Embodiments of the present disclosure provide a method for optimizing large language model conversation, an apparatus, a device, and a computer storage medium, which can at least solve the technical problem of how to make the responses of a large language model better meet user expectations.

In a first aspect, an embodiment of the present disclosure provides a method for optimizing large language model conversation, the method including:
in response to entering a current conversation turn, acquiring configuration information corresponding to the current conversation turn based on a type of the current conversation turn, wherein the configuration information includes response strategy configuration information and conversation turn switching strategy configuration information;
acquiring user input information corresponding to the current conversation turn;
determining whether the current conversation turn needs to be switched based on a conversation turn switching determination strategy corresponding to the conversation turn switching strategy configuration information;
in response to determining that the current conversation turn needs to be switched, determining a next conversation turn corresponding to the current conversation turn based on a conversation turn switching strategy corresponding to the conversation turn switching strategy configuration information, and entering the next conversation turn;
in response to determining that the current conversation turn does not need to be switched, generating a response task based on a response prompt corresponding to the response strategy configuration information and the user input information, and performing the response task calling a large language model to generate a response message; and outputting the response message in the current conversation turn.

In a second aspect, an embodiment of the present disclosure provides an apparatus for optimizing large language model conversation, the apparatus including:
an acquirer, configured to, in response to entering a current conversation turn, acquire configuration information corresponding to the current conversation turn based on a type of the current conversation turn, wherein the configuration information includes response strategy configuration information and conversation turn switching strategy configuration information; wherein the acquirer is further configured to acquire user input information corresponding to the current conversation turn;
a determiner, configured to determine whether the current conversation turn needs to be switched based on a conversation turn switching determination strategy corresponding to the conversation turn switching strategy configuration information;
a switcher, configured to, in response to determining that the current conversation turn needs to be switched, determine a next conversation turn corresponding to the current conversation turn based on a conversation turn switching strategy corresponding to the conversation turn switching strategy configuration information, and enter the next conversation turn;
a caller, configured to, in response to determining that the current conversation turn does not need to be switched, generate a response task based on a response prompt corresponding to the response strategy configuration information and the user input information, and perform the response task calling a large language model to generate a response message; and
an outputter, configured to output the response message in the current conversation turn.

In a third aspect, an embodiment of the present disclosure provides a device for optimizing large language model conversation, the device including: a processor and a memory storing computer program instructions; wherein the processor, when executing the computer program instructions, implements the aforementioned method for optimizing large language model conversation.

In a fourth aspect, an embodiment of the present disclosure provides a non-transitory computer-readable storage medium, on which computer program instructions are stored, wherein the computer program instructions, when executed by a processor, implement the aforementioned method for optimizing large language model conversation.

In a fifth aspect, an embodiment of the present disclosure provides a computer program product, wherein the instructions in the computer program product, when executed by a processor of an electronic device, cause the electronic device to perform the aforementioned method for optimizing large language model conversation.

### BRIEF DESCRIPTION OF DRAWINGS

To more clearly explain the technical solutions in the embodiments of the present disclosure, the accompanying drawings required for the embodiments of the present disclosure will be briefly introduced below. For those of ordinary skill in the art, other drawings can be obtained from these drawings without creative effort.

FIG. 1 is a schematic flowchart of a method for optimizing large language model conversation according to an embodiment of the present disclosure;

FIG. 2 is a schematic flowchart of a large language model conversation according to an embodiment of the present disclosure;

FIG. 3 is a schematic structural diagram of a apparatus for optimizing large language model conversation according to an embodiment of the present disclosure; and

FIG. 4 is a schematic structural diagram of a device for optimizing large language model conversation according to an embodiment of the present disclosure.

### DETAILED DESCRIPTION OF THE EMBODIMENTS

The features and exemplary embodiments of various aspects of the present disclosure will be described in detail below. To make the objectives, technical solutions, and advantages of the present disclosure clearer, the present disclosure will be further described in detail below with reference to the accompanying drawings and specific embodiments. It should be understood that the specific embodiments described herein are only for explaining the present disclosure and not for limiting it. For those skilled in the art, the present disclosure can be implemented without some of these specific details. The following description of the embodiments is merely to provide a better understanding of the present disclosure by showing examples of it.

It should be noted that, relational terms such as “first” and “second” are used merely to distinguish one object or operation from another, and do not necessarily require or imply any such actual relationship or order between these objects or operations. Moreover, the terms “includes,” “including,” or any other variation thereof are intended to cover a non-exclusive inclusion, such that a process, method, article, or device that includes a list of elements does not include only those elements but may include other elements not expressly listed or inherent to such process, method, article, or device. Without more constraints, an element preceded by “includes a . . . ” does not preclude the existence of additional identical elements in the process, method, article, or device that includes the element.

To solve the problems in the related art, embodiments of the present disclosure provide a method for optimizing large language model conversation, an apparatus, a device, and a computer storage medium. The method for optimizing large language model conversation provided by the embodiments of the present disclosure will be introduced first.

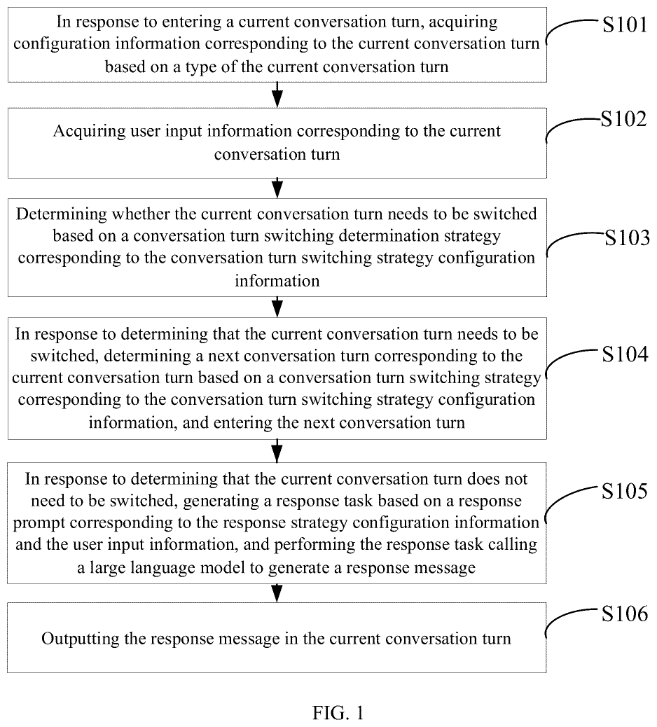

FIG. 1 is a schematic flowchart of a method for optimizing large language model conversation according to an embodiment of the present disclosure. As shown in FIG. 1, the method includes the following steps S101˜S106.

S101, in response to entering a current conversation turn, acquiring configuration information corresponding to the current conversation turn based on a type of the current conversation turn.

The configuration information includes at least response strategy configuration information and conversation turn switching strategy configuration information.

It can be understood that the type of the current conversation turn can be determined by methods combining user input, contextual information, preset conversation rules, and intent recognition algorithms, among others.

In some embodiments, natural language processing (NLP) technology can be used to analyze the user's text input and extract key information, including keyword extraction, intent recognition, and entity recognition.

If a user inputs “what medicine to take for a stomach ache,” the model can identify the keywords “stomach ache” and “what medicine to take,” which suggests that the current conversation type might be “medical knowledge support.”

In some embodiments, it can be determined based on contextual information, which can include previous user inputs, model responses, conversation topics, etc.

If the preceding conversation involved “what causes a stomach ache,” then the current turn might still be “medical knowledge support.”

In some embodiments, the current conversation type can be classified according to predefined rules or templates. When the user's input matches a specific pattern, the model will determine it as the corresponding conversation type. Several rules can be set. For example, if the user's input contains “inquiry” or “question,” the current turn is automatically determined to be “medical question consultation.”

In some embodiments, a pre-trained classification model (such as SVM, random forest, or a deep learning model) can be used to recognize the user's input intent. This model can learn different conversation types from a labeled dataset.

In some embodiments, the model's accuracy in identifying the current conversation type can be improved through continuous user interaction and feedback. If the model incorrectly determines the conversation type, the user can correct it through explicit statements.

If a user inserts “what does a certain doctor in the hospital specialize in” during a “medical question consultation” conversation, the model can adjust the conversation type in real-time to “medical question consultation” or “hospital doctor inquiry.”

In some embodiments, the conversation type can be comprehensively determined based on multimodal inputs (e.g., text, voice, images, etc.).

In a voice conversation, if the user's speech rate and emotion show a sense of urgency, the model might determine the conversation type as “emergency issue handling.”

It can be understood that at the beginning of a conversation, the type of the current conversation turn is determined (e.g., asking a question, solving a problem, casual chat, etc.), and then the related configuration information is extracted from a configuration file or database based on the type of the current conversation turn.

It can be understood that the configuration information can be pre-configured in the conversation backend by relevant operations and maintenance personnel. The entire conversation process can be divided into multiple stages, each corresponding to a specific task or goal. Specific steps and rules are defined for each stage (e.g., response strategy configuration information and/or conversation turn switching strategy configuration information), including how to handle user input, how to generate responses, when to switch to the next stage, etc. The conversation flow is dynamically adjusted based on user input and contextual information to ensure the fluency and effectiveness of the conversation.

In some embodiments, a conversation management engine (such as Rasa, Dialogflow, etc.) can be used to define and configure the conversation flow. Scripts can be written to implement the logic and rules of each stage, successfully configuring the configurable conversation flow into the conversation backend, thereby improving the flexibility and efficiency of the conversation system.

In other words, relevant operations and maintenance personnel can write corresponding scripts to implement different configuration information. The specific content of the configuration information varies with different actual application scenarios and will not be listed one by one here.

In some embodiments, configuration requests can be received through interfaces, command-line tools, graphical user interfaces, etc. After receiving a configuration request, the request content is parsed. Based on the parsed configuration information, the aforementioned conversation management engine is called to configure the large language model. It can be understood that the configuration can be done according to the actual configuration request, which is not limited here.

In some embodiments, corresponding configuration templates can also be preset. Each configuration template includes a set of corresponding configuration parameters. The specific configuration templates and parameters can be set according to the actual situation and are not limited here. For example, by performing semantic analysis on the input from operations and maintenance personnel, an appropriate configuration template is automatically selected, and the parameters in the configuration template are adjusted. For instance, based on the input “conversation turn 10” from operations and maintenance personnel, the conversation turn parameter is automatically updated from the initial parameter to “6.” As another example, by receiving the aforementioned configuration request, the content of the request can be automatically recognized based on the parsed configuration request, and the parameters in the configuration template can be adjusted accordingly. Finally, the updated configuration is applied to the large language model.

The response strategy configuration information and the conversation turn switching strategy configuration information determine how the model responds to user input and when to change the direction of the conversation. The following is a detailed explanation of these two types of configuration information.

Response strategy configuration information consists of rules and guidelines used to direct the large language model on how to generate responses. It can include response style, content, and form.

Response strategy configuration information includes response style, such as formal or informal, which determines whether to use a formal tone or a more friendly, colloquial style.

For example, formal: using honorifics and professional terminology. Informal: using colloquial language and concise sentences.

Response strategy configuration information includes the level of detail in the response, such as concise or detailed.

Concise or detailed indicates whether the response should be brief or provide a detailed explanation. Concise means directly answering the user's question. Detailed means providing background information and further suggestions.

Response strategy configuration information includes the type of emotional expression, such as neutral, encouraging, or warm, which can determine the emotion conveyed by the model in its response.

For example, neutral: no emotional words; encouraging: contains positive words, such as “That's great, we can solve this problem!”

Response strategy configuration information can be encoded as prompts or contextual information for the model, so that the generated response information conforms to the set style and goals. For example, specific prompts can be added when inputting to the model, making the generated response more aligned with these strategies.

Conversation turn switching strategy configuration information is used to identify when it is necessary to switch the conversation topic or turn, and how to perform this switch.

Whether the type or content of the user's input matches the theme of the current conversation turn is determined.

For example, if the user mentions “hospital opening hours,” it is switched to the “hospital strategy inquiry” turn. If the user does not respond for a long time, it is switched to the “end conversation” strategy.

When switching turns, information collected in the previous conversation can be retained for subsequent use.

As mentioned above, conversation turn switching strategy configuration information can also be implemented through natural language processing and machine learning algorithms. The large language model can learn patterns in user input and determine whether a turn switch is needed by analyzing context and semantics. For example, a method of tracking semantic slots can be used to identify the conversation intent and switch according to set rules.

In some embodiments, if the current conversation turn is “technical support,” the model retrieves from the configuration that this turn should use detailed technical terminology and should not switch the conversation as long as the user's problem is not resolved.

S102, acquiring user input information corresponding to the current conversation turn.

The user's text, image, voice, video, and other input data are acquired through an input interface (such as a chat box or voice recognition) and then parsed into structured data.

The user's needs and intent are understood by collecting the information entered by the user in the current conversation turn.

S103, determining whether the current conversation turn needs to be switched based on a conversation turn switching determination strategy corresponding to the conversation turn switching strategy configuration information.

In conjunction with the above, the degree of match between the user's input content and the current conversation goal is analyzed according to the conversation turn switching strategy, to check if the switching conditions are met. For example: switching the current conversation turn when the user repeatedly asks irrelevant questions, or when the conversation reaches a preset endpoint.

In some embodiments, an intent recognition model (such as BERT) can be used for intent classification, mapping the user's input to predefined intent categories.

In some embodiments, whether the user's input contains specific keywords or phrases is checked through rules or content-based matching. A keyword list is maintained to dynamically match user input with the current conversation topic.

In some embodiments, whether the user's input is consistent with historical information or the current topic is determined by analyzing the context of the current conversation. The user inputs and model responses from the past few turns are evaluated to determine if the user's intent has changed.

In some embodiments, the semantic similarity between the user's input and the current conversation goal can be calculated to determine their degree of match. Vector representations of the user's input and the conversation goal are acquired through word embeddings (such as Word2Vec, GloVe, etc.) or deep learning models, to calculate the cosine similarity. If the similarity is high, the current turn is continued; if the similarity is low, switching the conversation turn is considered.

In some embodiments, a user feedback mechanism can be introduced to adjust the determination of the conversation type through explicit feedback. The user explicitly expresses the intention to change the conversation topic in their input, as well as their satisfaction with the current conversation.

Whether the current conversation needs to be transferred is determined based on user input or other conditions.

S104, in response to determining that the current conversation turn needs to be switched, determining a next conversation turn corresponding to the current conversation turn based on a conversation turn switching strategy corresponding to the conversation turn switching strategy configuration information, and entering the next conversation turn.

Once it is determined that a conversation switch is needed, the model needs to determine a suitable new conversation turn.

It can be understood that each conversation turn usually represents a specific topic or task, such as “medical question consultation,” “hospital doctor inquiry,” “hospital strategy inquiry,” etc. The model selects the corresponding turn based on the user's context and input.

The conversation turn switching strategy can include the following: state recognition, to identify the state and topic of the current conversation; condition definition, to set the conditions for switching, including the content of the user's input, contextual information, etc.; and target state, to determine which new conversation turn to switch to based on the recognition and condition assessment.

In some embodiments, the conversation turn switching strategy can trigger a switch by setting a group of predefined rules based on keywords or patterns in the user's input. For example, using regular expressions or matching strategies to monitor specific patterns in the user's input.

In some embodiments, the conversation turn switching strategy can treat the conversation as a state machine, where each state represents a specific conversation turn, and defines the transition conditions from one state to another. For example, defining states and their transition rules in a state machine, and when user input is received, determining whether to perform state transition based on the current state and input.

The following describes this in the context of a specific scenario.

Scenario: The user needs to ask a medical-related question.

Initial Turn: The user asks, “What should I do for a stomach ache?” The model is in the “Medical Inquiry” turn.

User Input: “I want to know about a certain doctor's specialty.”

Keyword Matching: “doctor” and “specialty” are identified.

Context: The model checks if the previous conversation involved this topic and determines that the current topic is suitable for a switch.

Switching Strategy Decides: Current turn [Medical Inquiry]→Next turn [Hospital/Doctor Inquiry].

Enter Next conversation Turn: The model responds, “That doctor's specialty is . . . ” and formally switches to the “Hospital/Doctor Inquiry” turn.

S105, in response to determining that the current conversation turn does not need to be switched, generating a response task based on a response prompt corresponding to the response strategy configuration information and the user input information, and performing the response task calling a large language model to generate a response message.

A prompt is text used to guide the model to generate a specific type of response. It can be a question, a topic, or a description of the user's input context. Effective prompts usually include enough information for the model to understand the desired response content.

When generating a response, the model needs to integrate the user's input information, which is usually the context of the conversation. The user's input provides the model with the background information needed to generate a specific response.

Based on the prompt and the user's input information, a clear task is generated to guide the model in producing the desired response. This can be achieved by combining the prompt and the user's input.

The model first parses the meaning of the prompt to understand the type of information or topic the user wants. The model integrates the user's input into the context to ensure the generated response is relevant to the user's needs. Using its generation capabilities, the model combines the prompt, user input, and contextual information to generate a grammatically correct and semantically coherent response.

In some embodiments, to optimize the accuracy of the large language model's responses, one can use clear instructions and question formats, and optimize the design of prompts to ensure they are clear, specific, and less ambiguous. Clear instructions and question formats are preferably used.

For example, instead of prompting the user to ask, “Tell me about a certain doctor,” it's better to ask, “Please list what a certain doctor's specialty is.”

In some embodiments, including rich contextual information in the prompt can help the model better understand the user's intent.

For example, if the user has previously asked about a certain doctor, the prompt can include the department that doctor belongs to, allowing the model to quickly and accurately generate the corresponding response.

In some embodiments, specific examples can be provided to let the model understand the expected response style and content.

For example, a preset response template can be set up, to reply with the corresponding content according to this template.

In some embodiments, the direction of the model's responses can be continuously adjusted based on user feedback. For example, when a user is dissatisfied with the model's answer, the reason can be investigated and the prompt strategy can be improved accordingly by providing different types of prompts, comparing their effects, and identifying the most effective prompt strategy. S106, outputting the response message in the current conversation turn.

The generated response is conveyed to the user, completing one turn of the conversation, and the generated response is output through the user interface, ensuring the information is clear and readable.

In conjunction with the above, the model will reply, “The doctor's specialty is in the field of neurosurgery.” It then sends this to the results interface and awaits the user's response.

By acquiring the corresponding configuration information based on the type of the current conversation turn, the model can determine the appropriate response method for different types, which helps in accurately understanding the user's actual problem later; acquiring the user input information corresponding to the current conversation turn allows the model to accurately determine the user's intent and needs; based on the conversation turn switching determination strategy corresponding to the conversation turn switching strategy configuration information, it determines whether the current conversation turn needs to be switched, ensuring the conversation can switch to the next turn at the appropriate time and avoiding continuing the current turn at an unsuitable moment; if a switch is needed, it determines the next conversation turn corresponding to the current one based on the conversation turn switching determination strategy and enters the next turn, ensuring a smooth transition and maintaining the conversation's coherence and logic; if no switch is needed, it generates a response based on the response prompt and user input information corresponding to the response strategy configuration, ensuring the model can generate a response that meets the requirements of the current conversation turn and improving the accuracy and relevance of the response; the generated response is returned to the user, completing the interaction for the current conversation turn and making the model's response better align with the user's expectations.

In other words, by acquiring user input and configuration information, the model can accurately understand the user's needs. The conversation turn switching strategy ensures that the conversation can switch to the next turn at the appropriate time, maintaining coherence and logic. The response strategy configuration and the large language model generate responses that meet the requirements of the current conversation turn, improving accuracy and relevance. Through smooth conversation state transitions and high-quality responses, the user experience can be enhanced.

In an embodiment, generating a response task based on the response prompt and user input information corresponding to the response strategy configuration, and calling the large language model to execute the response task to generate a response, includes: determining whether the user input information satisfies a summarization strategy trigger condition corresponding to the response strategy configuration information; in response to determining that the user input information satisfies the summarization strategy trigger condition, generating a summarization conversation task based on the user input information, a conversation context of the user input information in the current conversation turn, and a summarization conversation prompt corresponding to the response strategy configuration information, and performing the summarization conversation task through the large language model to generate a summarization response message.

A summarization strategy trigger condition is a preset rule or condition used to decide when the summarization strategy should be invoked. For example, a summarization can be triggered when the user's query involves a series of information.

In some embodiments, techniques like keyword matching, topic modeling, or semantic analysis can be used to assess whether the user's input involves multiple topics or complex content suitable for summarization.

The user input information, conversation context, and the corresponding summarization conversation prompt are integrated into a structured task request. For instance, this could be formatting the information into a concise instruction: “Please summarize all the information from the user in the conversation.”

The large language model is called, which performs inference and generation through the provided summarization conversation task (including the prompt, user input, and context). The corresponding code could be a direct API call to interact with the model. Through the model's generation capabilities, a condensed summarization is produced, integrating the user's input and historical conversation content, ensuring the information is accurate and easy to understand.

The generated summarization information is feed back to the user, typically by formatting the model's output into readable sentences or paragraphs and displaying it to the user.

By determining whether the user input information meets the summarization strategy trigger conditions, the system can accurately identify if the user needs a conversation summarization; by using the user input information, conversation context, and summarization conversation prompt, it generates a summarization task that fits the current conversation context and uses the large language model to generate an accurate and coherent summarization response, enhancing the user experience and helping the user quickly grasp the key content of the conversation.

In an embodiment, generating a response task based on the response prompt and user input information corresponding to the response strategy configuration, and calling the large language model to execute the response task to generate a response, further includes: determining whether the user input information satisfies a follow-up question strategy trigger condition corresponding to the response strategy configuration information; and in response to determining that the user input information satisfies the follow-up question strategy trigger condition corresponding to the response strategy configuration information, generating an intelligent follow-up question response task based on the user input information, a conversation context of the user input information in the current conversation turn, and an intelligent follow-up question prompt corresponding to the response strategy configuration information, and performing the intelligent follow-up question response task through the large language model to generate an intelligent follow-up question response message.

The follow-up question strategy trigger condition refers to the specific conditions used to determine in what context a follow-up question strategy should be invoked. For example, if the user's answer is vague, the model might request more information.

A intelligent follow-up question prompt is a prompt used to guide the large language model in generating a follow-up question, including specific questions or instructions that clarify the required follow-up content.

Natural Language Processing (NLP) techniques such as keyword extraction, sentiment analysis, or intent recognition can be used to determine whether the user's input meets the preset conditions for a follow-up question. For example, a follow-up should be triggered when ambiguity, lack of detail, or an incomplete response is detected. This ensures that follow-up questions are asked in appropriate scenarios, thereby increasing the effectiveness of the conversation, helping to acquire more detailed user information, and promoting a smooth subsequent conversation.

The historical information of the current conversation turn (user's input and model's responses) is used as context, keeping this information structured, for example, by passing it as a list or short text.

Context can provide the necessary background for the model, helping it understand the user's intent and past discussions to generate highly relevant and targeted follow-up questions.

Based on the user input information, conversation context, and the intelligent follow-up question prompt corresponding to the follow-up strategy, this information is constructed into a formatted task request. For example, an instruction is constructed like, “Please ask the user for more information about their request,” so the large language model receives a specific instruction, clearly guiding the follow-up question it needs to ask.

The large language model is used to generate a follow-up question response by calling its API and passing the constructed follow-up task. The model will generate an appropriate question based on the input content. The generated follow-up can be open-ended or specific, aiming to gather more information, help the model better understand the user's needs, and drive the conversation deeper. Format the follow-up information generated by the model into easy-to-read and understandable text and display it to the user.

By determining whether the user input information meets the follow-up question strategy trigger conditions, the system can accurately identify if the user needs further clarification; by using the user input information, conversation context, and intelligent follow-up question prompt, it generates a intelligent follow-up task that fits the current conversation context. Through effective follow-up questions, it guides the user to provide more information, enabling the large language model to make more precise responses while also enhancing the user's sense of engagement and satisfaction.

In an embodiment, determining whether the current conversation turn needs to be switched based on the conversation turn switching determination strategy corresponding to the conversation turn switching strategy configuration information includes: determining a preset number of conversation turns corresponding to the type of the current conversation turn according to the conversation turn switching strategy configuration information; in response to determining that the current conversation turn is greater than or equal to a preset number of conversation turns, determining that the current conversation turn needs to be switched; and in response to determining that the current conversation turn is less than the preset number of conversation turns, determining that the current conversation turn does not need to be switched.

A conversation turn refers to a complete exchange between the user and the model, i.e., the user inputs information once and receives a response from the model. Each turn of the conversation can be seen as an interaction loop.

The conversation turn switching strategy configuration information includes rules for different conversation scenarios, specifying that when the number of conversation turns reaches a certain number, it needs to switch to another conversation type or topic.

Preset number of conversation turns refer to: in a specific conversation type, a pre-configured maximum number of conversation turns. If the number of conversation turns exceeds this number, the model will need to make a switch.

The large language model loads a configuration file when starting a conversation, which contains the preset number of conversation turns for different conversation types (such as hospital strategy inquiry, medical inquiry, etc.). By determining the type of the current conversation, it retrieves the corresponding preset number. The maximum number of turns allowed in the current conversation state is determined to help determine later whether a turn switch is necessary.

Whether the current conversation turn is greater than or equal to a preset number of turns is determined, using a counter to record the number of turns in the current session. This counter increments with each user input and model response. Then, the counter's value is compared with the preset number of turns.

When the current conversation turn is greater than or equal to the preset number, the large language model marks that a switch is needed; when the current turn is less than the preset number, it marks that no switch is needed.

When a switch is needed, it might guide the user to a new topic or provide new consultation guidance; when no switch is needed, it maintains the current topic's dynamic for continued interaction.

By switching conversation turns at the right time, the efficiency and orderliness of the conversation are ensured, avoiding ineffective repetition or topic deviation. This ensures that the user can acquire the required information or solve their problem within a limited number of turns, enhancing the user experience.

In an embodiment, based on the conversation turn switching strategy configuration, a corresponding determination strategy is used to determine whether the current turn needs to be switched. This determination strategy further includes: determining an information collection task corresponding to the type of the current conversation turn according to the conversation turn switching strategy configuration information; extracting target information from the conversation context in the current conversation turn according to the information collection task, and calculating an information collection completion degree; in response to determining that the information collection completion degree is greater than or equal to a preset completion degree, determining that the current conversation turn needs to be switched; and in response to determining that the information collection completion degree is less than the preset completion degree, determining that the current conversation turn does not need to be switched.

The information collection task is a specific goal the model needs to achieve in a particular conversation turn, such as collecting specific user information (e.g., name, age, past medical history, needs, etc.).

The information collection completion rate can quantify the ratio between the information already collected and the expected amount of information. It is usually expressed as a percentage to measure the adequacy of the information collection.

The preset completion rate is a standard set by the model in advance, indicating the completion threshold that must be reached during an information collection task to decide whether the conversation needs to be switched.

When the model starts, it loads the conversation turn switching strategy configuration, which contains the information collection tasks to be completed for the current conversation type. By parsing the context and type of the current conversation, the model can identify the specific information domains needed and clarify the goal of the current conversation. This allows the model to effectively collect information throughout the conversation and ensure the user's needs are fully understood and addressed.

In some embodiments, target information can be extracted from the conversation context of the current turn. The model analyzes the historical context of the current conversation and uses natural language processing techniques (such as entity recognition, keyword extraction, etc.) to extract target information related to the information collection task from the user's input. The model compares the amount of extracted information with the predefined total amount of necessary information to calculate a completion percentage. For example, by dividing the number of collected information items by the number of expected information items (e.g., number of items obtained/number of items required×100%).

Quantifying the state of information collection provides a basis for subsequently evaluating whether a conversation turn switch is needed. By calculating the completion rate, the model can promptly assess the conversation's progress and decide whether to continue the current task or switch to a new topic.

The calculated information collection completion rate is compared with the preset completion rate. If the completion rate is greater than or equal to the preset rate, the model marks that a switch is needed; if it is less, it marks that no switch is needed.

Based on the type of the current conversation turn, the specific information content to be collected is clarified, as different conversation types may require collecting different types of information. This defines the specific information to be collected for each conversation type, ensuring the conversation proceeds with a clear focus. It also allows for a timely conclusion after sufficient information is gathered, avoiding lengthy and inefficient conversations.

In an embodiment, the in response to determining that the current conversation turn needs to be switched, determining a next conversation turn corresponding to the current conversation turn based on a conversation turn switching strategy corresponding to the conversation turn switching strategy configuration information, and entering the next conversation turn, includes:
determining a prompt corresponding to the next conversation turn according to the conversation turn switching strategy configuration information; and
proceeding with the next conversation turn according to the prompt corresponding to the next conversation turn.

The prompt for the next conversation turn refers to the statements or questions used to guide the user in the new turn. These prompts are typically used to engage the user and set the direction of the conversation.

When loading the conversation turn switching strategy, the prompt corresponding to each turn is recorded. When it is determined that a switch to a new conversation turn is needed, the model retrieves a prompt that matches the current conversational context. This may involve querying a structured data source, such as a database or configuration file, to find the appropriate prompt content.

This clarifies how to guide the user next, setting expectations and encouraging participation in a new topic or direction. These prompts can help the user better understand the upcoming conversation content, increasing their sense of engagement.

Once the prompt for the next conversation turn is determined, the model will construct a new conversational response that includes the prompt obtained from the previous step. Using natural language generation technology, the model can transform these prompts into complete and easily understandable sentences and present them to the user.

By generating and displaying the prompt, the model guides the user into a new round of conversation, a process that helps ensure the fluency and effectiveness of the conversation. The user will be encouraged to respond to new questions or engage in further discussion, thereby enhancing the depth and breadth of the conversation.

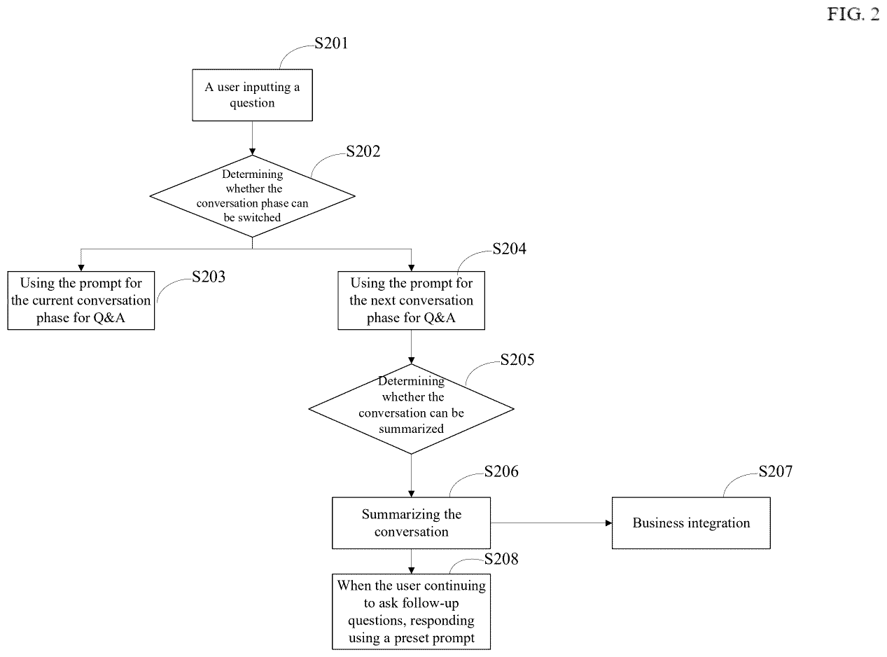

Next, the method for optimizing large language model conversation mentioned above will be introduced holistically with reference to FIG. 2.

FIG. 2 is a schematic flowchart of a large language model conversation according to an embodiment of the present disclosure

As shown in FIG. 2, the conversation flow includes the following steps S201˜S208.

S201, a user inputting a question.

A Q&A is conducted based on the first prompt.

S202, determining whether the conversation phase can be switched.

S203, when it cannot be switched, using the prompt for the current conversation phase for Q&A.

S204, when it can be switched, using the prompt for the next conversation phase for Q&A.

S205, determining whether the conversation can be summarized.

S206, summarizing the conversation.

S207, business integration.

The collected conversation data is organized into business data and passed to the corresponding business systems or personnel.

S208, when the user continuing to ask follow-up questions, responding using a preset prompt.

By providing a configurable conversation flow, the above steps are configured into the conversation backend according to the process. After the user inputs a question, the program will respond according to the steps, making the conversation steps controllable.

For example, the configuration for a medical and health consultation is implemented as follows:
1. The user asks, “What should I do about a stomach ache?”
2. The system replies based on the first prompt.
3. The user continues to consult after receiving the information.
4. The program will determine whether the user's input meets the requirements for switching prompts, for example, by binding the number of conversation turns and information collection logic to the prompt in the configuration backend.

After the user's input, the prompt determines if the current number of conversation turns meets the configured requirement. If the turn count is met, it switches to the next prompt for the reply.

After the user's input, the information collector analyzes the historical conversation, extracts the necessary information for collection, and compares it against the configuration requirements for the information points on the prompt. If the required information has been collected, it switches to the next prompt for the reply.

In some embodiments, the conversation turn switching strategy configuration information mentioned above can include configuration requirements corresponding to the information to be collected.

The information collector will review the previous conversation records and, based on the configuration requirements for the information to be collected, identify and extract specific types of information.

It is understandable that a lot of information may be extracted. The information collector will filter it according to certain criteria to remove irrelevant or redundant information. The filtering criteria can include the relevance, accuracy, and importance of the information.

The information collector will compare the extracted information with these prompts to ensure that the extracted information meets the requirements. For example, if the prompt is “doctor's specialty,” the information collector will filter out all sentences that mention the doctor's specialty.

When the requirements for summarizing the conversation are met, the system will collect user information based on the preset summarization prompt, such as: user gender, age, height, weight, history of present illness, past medical history, family history, allergy history, etc.
5. After the information is collected, business integration can be performed, such as generating a medical record.
6. When the user continues to ask follow-up questions, a fallback prompt is used to continue the reply.
7. Depending on the type of conversation workflow, different prompts are used for replies. For Q&A type prompts, the user's question is analyzed and answered. For information collection type prompts, user information is collected and questions are asked until the information collection requirements are met, such as completion of information collection, hitting a specified logic, etc.

For example, if the purpose of the user's conversation prompt is for a patient's health consultation, then the prompt is a Q&A type. Whatever the patient inputs, the prompt will not ask follow-up questions but will only answer the patient's question. If the user's conversation type prompt is for symptom consultation, the prompt will ask the patient questions and then collect information.

The user information from the conversation process is structured. Based on the configuration requirements for the information to be collected in the prompt, the information points hit in the context of the conversation are extracted, and the information points are structured into the required data.

That is to say, in conjunction with the configuration requirements for collecting information mentioned above, the extracted information points are organized into structured data according to a predetermined format. For example, the medical record data generated in the above case can be structured in json format.

```
{
  “User Gender”: “Large Language Model Conversation Optimization”,
  “User Age”: “Large Language Model Conversation Optimization”,
  “User Height”: “Large Language Model Conversation Optimization”,
  “User Weight”: “Large Language Model Conversation Optimization”,
  “History of Present Illness”: “Large Language Model Conversation Optimization”,
  “Past Medical History”: “Large Language Model Conversation Optimization”,
  “Family History”: “Large Language Model Conversation Optimization”,
  “Allergy History”: “Large Language Model Conversation Optimization”
}
```

Using this data for business integration, it can be displayed as a hospital medical record.

The model intelligently asks follow-up questions based on the conversation context. For example, after the business data summarization is completed, a conversation phase marker will be generated. If the user continues to ask questions, the system will continue to reply to the user based on the type of follow-up prompt.

If it is a Q&A type prompt, it will answer the user's question based on their input. For example, in a health consultation conversation workflow, if the user asks “What should I do about a stomach ache?”, the prompt will analyze the causes of the stomach ache based on the user's question and provide health advice and precautions.

If it is an information collection type prompt, it will ask the user questions based on their input. For example, in a disease diagnosis conversation workflow, if the user asks “What should I do about a stomach ache?”, the prompt, based on the user's question and combined with an internal disease knowledge base, will ask the user “How long has the stomach ache lasted and are there any accompanying symptoms?”, forming a Q&A exchange with the user until enough information is collected to identify a possible disease, and then provide a preliminary diagnosis.

The application scenarios of this method will be introduced below with specific examples.

User input: “I want to know about physical examinations.”

Model response: “Which physical examination items would you like to know about?”

User answer: “I want to know about liver function, kidney function, and an electrocardiogram.”

The model asks for the user's basic information based on a preset prompt to determine if their current physical condition supports undergoing these examination items.

The model collects information and structures it, generating json format data:

```
  {
  “Basic Information”: {
    “User ID”: “Large Language Model conversation Optimization”,
    “User Gender”: “Large Language Model conversation Optimization”,
    “User Age”: “Large Language Model conversation Optimization”,
  },
  “Examination Items”: [“Liver Function”, “Kidney Function”, “Electrocardiogram”]
}
```

User input: “Precautions for the physical examination.”

The model continues to provide relevant information.

The model identifies the user's intent through prompt configuration, collects the user's basic information and the desired examination items, and generates a physical examination checklist. The user can directly book an offline physical examination based on the checklist. Based on the method for optimizing large language model conversation provided in the above embodiments, the present disclosure correspondingly provides a specific implementation of a apparatus for optimizing large language model conversation. Please refer to the following embodiments.

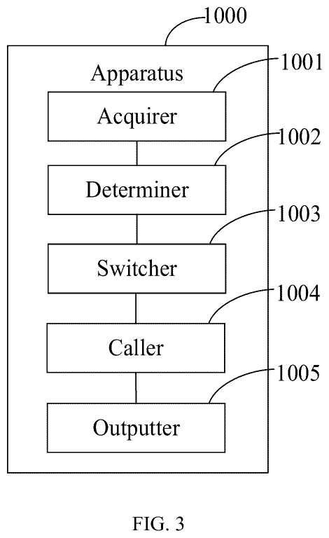

FIG. 3 is a schematic structural diagram of a apparatus for optimizing large language model conversation according to an embodiment of the present disclosure.

As shown in FIG. 3, the apparatus 1000 for optimizing large language model conversation includes the following components:
an acquirer 1001, configured to, in response to entering a current conversation turn, acquire configuration information corresponding to the current conversation turn based on a type of the current conversation turn, wherein the configuration information comprises response strategy configuration information and conversation turn switching strategy configuration information;
wherein the acquirer 1001 is further configured to acquire user input information corresponding to the current conversation turn;
a determiner 1002, configured to determine whether the current conversation turn needs to be switched based on a conversation turn switching determination strategy corresponding to the conversation turn switching strategy configuration information;
a switcher 1003, configured to, in response to determining that the current conversation turn needs to be switched, determine a next conversation turn corresponding to the current conversation turn based on a conversation turn switching strategy corresponding to the conversation turn switching strategy configuration information, and enter the next conversation turn;
a caller 1004, configured to, in response to determining that the current conversation turn does not need to be switched, generate a response task based on a response prompt corresponding to the response strategy configuration information and the user input information, and perform the response task calling a large language model to generate a response message; and
an outputter 1005, configured to output the response message in the current conversation turn.

In some embodiments, the aforementioned caller 1004 is also for generating a response task based on the response prompt corresponding to the response strategy configuration information and the user input information, calling the large language model to perform the response task, and generating response information, which includes: determining whether the user input information satisfies a summarization strategy trigger condition corresponding to the response strategy configuration information; in response to determining that the user input information satisfies the summarization strategy trigger condition corresponding to the response strategy configuration information, generating a summarization conversation task based on the user input information, a conversation context of the user input information in the current conversation turn, and a summarization conversation prompt corresponding to the response strategy configuration information, and performing the summarization conversation task through the large language model to generate a summarization response message.

By determining whether the user input information meets the summarization strategy trigger condition, the system can accurately identify whether the user has a need for conversation summarization; by using the user input information, conversation context, and summarization prompt, it generates a summarization task that fits the current conversation context and uses the large language model to generate an accurate and coherent summarization response, enhancing the user experience and helping the user quickly understand the key content of the conversation.

In some embodiments, the aforementioned caller 1004 is also for generating a response task based on the response prompt corresponding to the response strategy configuration information and the user input information, calling the large language model to perform the response task, and generating response information, which further includes: determining whether the user input information satisfies a follow-up question strategy trigger condition corresponding to the response strategy configuration information; and in response to determining that the user input information satisfies the follow-up question strategy trigger condition corresponding to the response strategy configuration information, generating an intelligent follow-up question response task based on the user input information, a conversation context of the user input information in the current conversation turn, and an intelligent follow-up question prompt corresponding to the response strategy configuration information, and performing the intelligent follow-up question response task through the large language model to generate an intelligent follow-up question response message.

By determining whether the user input information meets the follow-up question strategy trigger condition, the system can accurately identify whether the user has a need for further clarification; by using the user input information, conversation context, and intelligent follow-up question prompt, it generates a intelligent follow-up question task that fits the current conversation context. Through effective follow-up questions, it guides the user to provide more information, enabling the large language model to provide a more precise response, while also enhancing the user's sense of engagement and satisfaction.

In some embodiments, the aforementioned determiner 1002 is also for determining whether the current conversation turn needs to be switched based on the conversation turn switching determination strategy corresponding to the conversation turn switching strategy configuration information, which includes: determining a preset number of conversation turns corresponding to the type of the current conversation turn according to the conversation turn switching strategy configuration information; in response to determining that the current conversation turn is greater than or equal to a preset number of conversation turns, determining that the current conversation turn needs to be switched; and in response to determining that the current conversation turn is less than the preset number of conversation turns, determining that the current conversation turn does not need to be switched.

By switching conversation turns at the appropriate time, the efficiency and orderliness of the conversation are ensured, avoiding ineffective repetition or deviation from the topic, which ensures that the user can acquire the required information or solve problems within a limited number of turns, thus enhancing the user experience.

In some embodiments, the aforementioned determiner 1002 is also for determining whether the current conversation turn needs to be switched based on the conversation turn switching determination strategy corresponding to the conversation turn switching strategy configuration information, which also includes: determining an information collection task corresponding to the type of the current conversation turn according to the conversation turn switching strategy configuration information; extracting target information from the conversation context in the current conversation turn according to the information collection task, and calculating an information collection completion degree; in response to determining that the information collection completion degree is greater than or equal to a preset completion degree, determining that the current conversation turn needs to be switched; and in response to determining that the information collection completion degree is less than the preset completion degree, determining that the current conversation turn does not need to be switched.

Based on the type of the current conversation turn, the content of the information to be collected is clarified, because different conversation types may require collecting different types of information; the specific information to be collected for each conversation type is defined, ensuring that the conversation proceeds with a clear purpose; the conversation is ended in a timely manner after sufficient information has been collected, avoiding lengthy and inefficient conversations.

In some embodiments, the aforementioned switcher 1003 is also for, when it is determined that the current conversation turn needs to be switched, determining the next conversation turn corresponding to the current conversation turn based on the conversation turn switching strategy corresponding to the conversation turn switching strategy configuration information, and entering the next conversation turn, which includes: determining the prompt corresponding to the next conversation turn according to the conversation turn switching strategy configuration information; and proceeding to the next conversation turn according to the prompt corresponding to the next conversation turn.

By generating and displaying a prompt, the model guides the user into a new round of conversation, a process that helps ensure the fluency and effectiveness of the conversation. The user will be encouraged to respond to new questions or engage in further discussion, thereby enhancing the depth and breadth of the conversation.

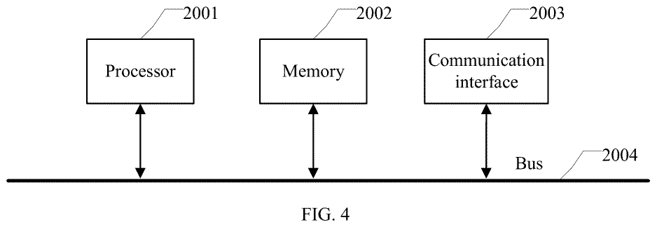

FIG. 4 is a schematic structural diagram of a device for optimizing large language model conversation according to an embodiment of the present disclosure.

The device for optimizing large language model conversation can include a processor 2001 and a memory 2002 storing computer program instructions.

Specifically, the aforementioned processor 2001 can include a central processing unit (CPU), or an Application Specific Integrated Circuit (ASIC), or can be configured as one or more integrated circuits for implementing the embodiments of the present disclosure.

The memory 2002 can include mass storage for data or instructions. By way of example and not limitation, the memory 2002 may include a Hard Disk Drive (HDD), a floppy disk drive, flash memory, an optical disc, a magneto-optical disc, a magnetic tape, or a Universal Serial Bus (USB) drive, or a combination of two or more of these. Where appropriate, the memory 2002 may include removable or non-removable (or fixed) media. Where appropriate, the memory 2002 may be internal or external to the integrated gateway disaster recovery device. In a particular embodiment, the memory 2002 is a non-volatile solid-state memory.

In an embodiment, the memory may include a read-only memory (ROM), random access memory (RAM), disk storage media devices, optical storage media devices, flash memory devices, electrical, optical, or other physical/tangible memory storage devices. Therefore, generally, the memory includes one or more tangible (non-transitory) computer-readable storage media (e.g., a memory device) encoded with software including computer-executable instructions, and when the software is executed (e.g., by one or more processors), it is operable to perform the operations described with reference to the methods according to the first aspect of the present disclosure.

The processor 2001 implements any of the methods for optimizing large language model conversation in the above embodiments by reading and executing the computer program instructions stored in the memory 2002.

In an embodiment, the device for optimizing large language model conversation may also include a communication interface 2003 and a bus 2000. As shown in FIG. 4, the processor 2001, the memory 2002, and the communication interface 2003 are connected and communicate with each other via the bus 2000.

The communication interface 2003 is mainly used to implement communication between the various modules, apparatuses, units, and/or devices in the embodiments of the present disclosure.

The bus 2000 includes hardware, software, or both, coupling the components of the device for optimizing large language model conversation to each other. By way of example and not limitation, the bus may include an Accelerated Graphics Port (AGP) or other graphics bus, an Enhanced Industry Standard Architecture (EISA) bus, a Front-Side Bus (FSB), a HyperTransport (HT) interconnect, an Industry Standard Architecture (ISA) bus, an InfiniBand interconnect, a Low Pin Count (LPC) bus, a memory bus, a Micro Channel Architecture (MCA) bus, a Peripheral Component Interconnect (PCI) bus, a PCI-Express (PCI-X) bus, a Serial Advanced Technology Attachment (SATA) bus, a Video Electronics Standards Association Local (VLB) bus, or other suitable bus, or a combination of two or more of the above. Where appropriate, the bus 2000 may include one or more buses. Although the embodiments of the present disclosure describe and illustrate specific buses, the present disclosure may contemplate any suitable bus or interconnect.

Additionally, in conjunction with the method for optimizing large language model conversation in the above embodiments, the embodiments of the present disclosure may provide a non-transitory computer storage medium to implement them. The computer storage medium stores computer program instructions; when the computer program instructions are executed by a processor, they implement any of the methods in the above embodiments.

An embodiment of the present disclosure further provides a computer program product, which includes a computer program. When the computer program is executed by a processor, it implements any methods for optimizing large language model conversation in the above embodiments.

It should be clarified that the present disclosure is not limited to the specific configurations and processes described above and shown in the drawings. For the sake of brevity, detailed descriptions of known methods are omitted here. In the above embodiments, several specific steps are described and shown as examples. However, the method process of the present disclosure is not limited to the specific steps described and shown. Those skilled in the art can make various changes, modifications, and additions, or change the order between steps, after understanding the principle of the present disclosure.

The functional blocks shown in the structural block diagrams described above may be implemented as hardware, software, firmware, or a combination thereof. When implemented in hardware, it can be, for example, an electronic circuit, an application specific integrated circuit (ASIC), appropriate firmware, a plug-in, a function card, and so on. When implemented in software, the elements of the present disclosure are programs or code segments used to perform the required tasks. The program or code segments can be stored in a machine-readable medium or transmitted over a transmission medium or communication link via a data signal carried in a carrier wave. A “machine-readable medium” may include any medium capable of storing or transmitting information. Examples of machine-readable media include electronic circuits, semiconductor memory devices, read-only memory (ROM), flash memory, erasable read only memory (EROM), floppy disks, compact disc read-only memory (CD-ROM), optical disks, hard disks, fiber optic media, radio frequency (RF) links, and so on. The code segments may be downloaded via computer networks such as the Internet, intranets, etc.

It also needs to be stated that the exemplary embodiments mentioned in the present disclosure describe some methods or systems based on a series of steps or devices. However, the present disclosure is not limited to the order of the steps described above, that is, the steps can be executed in the order mentioned in the embodiments, or in a different order from the embodiments, or several steps can be executed simultaneously.

The various aspects of the present disclosure have been described above with reference to flowcharts and/or block diagrams of methods, apparatuses (systems), and computer program products according to embodiments of the present disclosure. It should be understood that each block in the flowcharts and/or block diagrams, and combinations of blocks in the flowcharts and/or block diagrams, can be implemented by computer program instructions. These computer program instructions may be provided to the processor of a general-purpose computer, a special-purpose computer, or other programmable data processing apparatus to produce a machine, such that these instructions, when executed via the processor of the computer or other programmable data processing apparatus, enable the implementation of the functions/actions specified in one or more blocks of the flowchart and/or block diagram. Such a processor may be, but is not limited to, a general-purpose processor, a special-purpose processor, a special application processor, or a field-programmable logic circuit. It can also be understood that each block in the block diagrams and/or flowcharts, and combinations of blocks in the block diagrams and/or flowcharts, can also be implemented by dedicated hardware that performs the specified functions or actions, or can be implemented by a combination of dedicated hardware and computer instructions.

The foregoing are only specific embodiments of the present disclosure. It can be clearly understood by those skilled in the art that, for the convenience and brevity of description, the specific working processes of the systems, modules, and units described above can be referred to the corresponding processes in the foregoing method embodiments, and will not be repeated here. It should be understood that the scope of protection of the present disclosure is not limited to this. Any person skilled in the art can easily conceive of various equivalent modifications or replacements within the technical scope disclosed by the present disclosure, and these modifications or replacements should be covered within the scope of protection of the present disclosure.

What is claimed is:

1. A method for optimizing large language model conversation, the method comprising:
in response to entering a current conversation turn, acquiring configuration information corresponding to the current conversation turn based on a type of the current conversation turn, wherein the configuration information comprises response strategy configuration information and conversation turn switching strategy configuration information;
acquiring user input information corresponding to the current conversation turn;
determining whether the current conversation turn needs to be switched based on a conversation turn switching determination strategy corresponding to the conversation turn switching strategy configuration information;
in response to determining that the current conversation turn needs to be switched, determining a next conversation turn corresponding to the current conversation turn based on a conversation turn switching strategy corresponding to the conversation turn switching strategy configuration information, and entering the next conversation turn;
in response to determining that the current conversation turn does not need to be switched, generating a response task based on a response prompt corresponding to the response strategy configuration information and the user input information, and performing the response task calling a large language model to generate a response message; and
outputting the response message in the current conversation turn.

2. The method according to claim 1, wherein the generating a response task based on a response prompt corresponding to the response strategy configuration information and the user input information, and performing the response task calling a large language model to generate a response message, comprises:
determining whether the user input information satisfies a summarization strategy trigger condition corresponding to the response strategy configuration information;
in response to determining that the user input information satisfies the summarization strategy trigger condition corresponding to the response strategy configuration information, generating a summarization conversation task based on the user input information, a conversation context of the user input information in the current conversation turn, and a summarization conversation prompt corresponding to the response strategy configuration information, and performing the summarization conversation task through the large language model to generate a summarization response message.

3. The method according to claim 1, wherein the generating a response task based on a response prompt corresponding to the response strategy configuration information and the user input information, and performing the response task calling a large language model to generate a response message, further comprises:
determining whether the user input information satisfies a follow-up question strategy trigger condition corresponding to the response strategy configuration information; and
in response to determining that the user input information satisfies the follow-up question strategy trigger condition corresponding to the response strategy configuration information, generating an intelligent follow-up question response task based on the user input information, a conversation context of the user input information in the current conversation turn, and an intelligent follow-up question prompt corresponding to the response strategy configuration information, and performing the intelligent follow-up question response task through the large language model to generate an intelligent follow-up question response message.

4. The method according to claim 1, wherein the determining whether the current conversation turn needs to be switched based on a conversation turn switching determination strategy corresponding to the conversation turn switching strategy configuration information, comprises:
determining a preset number of conversation turns corresponding to the type of the current conversation turn according to the conversation turn switching strategy configuration information;
in response to determining that the current conversation turn is greater than or equal to a preset number of conversation turns, determining that the current conversation turn needs to be switched; and
in response to determining that the current conversation turn is less than the preset number of conversation turns, determining that the current conversation turn does not need to be switched.

5. The method according to claim 4, wherein the determining whether the current conversation turn needs to be switched based on a conversation turn switching determination strategy corresponding to the conversation turn switching strategy configuration information, further comprises:
determining an information collection task corresponding to the type of the current conversation turn according to the conversation turn switching strategy configuration information;
extracting target information from the conversation context in the current conversation turn according to the information collection task, and calculating an information collection completion degree;
in response to determining that the information collection completion degree is greater than or equal to a preset completion degree, determining that the current conversation turn needs to be switched; and
in response to determining that the information collection completion degree is less than the preset completion degree, determining that the current conversation turn does not need to be switched.

6. The method according to claim 1, wherein the in response to determining that the current conversation turn needs to be switched, determining a next conversation turn corresponding to the current conversation turn based on a conversation turn switching strategy corresponding to the conversation turn switching strategy configuration information, and entering the next conversation turn, comprises:
determining a prompt corresponding to the next conversation turn according to the conversation turn switching strategy configuration information; and
proceeding with the next conversation turn according to the prompt corresponding to the next conversation turn.

7. An apparatus for optimizing large language model conversation, the apparatus comprising:
an acquirer, configured to, in response to entering a current conversation turn, acquire configuration information corresponding to the current conversation turn based on a type of the current conversation turn, wherein the configuration information comprises response strategy configuration information and conversation turn switching strategy configuration information; wherein the acquirer is further configured to acquire user input information corresponding to the current conversation turn;
a determiner, configured to determine whether the current conversation turn needs to be switched based on a conversation turn switching determination strategy corresponding to the conversation turn switching strategy configuration information;
a switcher, configured to, in response to determining that the current conversation turn needs to be switched, determine a next conversation turn corresponding to the current conversation turn based on a conversation turn switching strategy corresponding to the conversation turn switching strategy configuration information, and enter the next conversation turn;
a caller, configured to, in response to determining that the current conversation turn does not need to be switched, generate a response task based on a response prompt corresponding to the response strategy configuration information and the user input information, and perform the response task calling a large language model to generate a response message; and
an outputter, configured to output the response message in the current conversation turn.

8. A non-transitory computer-readable storage medium, on which computer program instructions are stored, wherein the computer program instructions, when executed by a processor, cause the processor to perform acts comprising:
in response to entering a current conversation turn, acquiring configuration information corresponding to the current conversation turn based on a type of the current conversation turn, wherein the configuration information comprises response strategy configuration information and conversation turn switching strategy configuration information;
acquiring user input information corresponding to the current conversation turn;
determining whether the current conversation turn needs to be switched based on a conversation turn switching determination strategy corresponding to the conversation turn switching strategy configuration information;
in response to determining that the current conversation turn needs to be switched, determining a next conversation turn corresponding to the current conversation turn based on a conversation turn switching strategy corresponding to the conversation turn switching strategy configuration information, and entering the next conversation turn;
in response to determining that the current conversation turn does not need to be switched, generating a response task based on a response prompt corresponding to the response strategy configuration information and the user input information, and performing the response task calling a large language model to generate a response message; and
outputting the response message in the current conversation turn.
